(19) United States
(12) Patent Application Publication
(10) Pub. No.: US 2021/0267919 A1
(43) Pub. Date: Sep. 2, 2021

(54) STABILIZED COATED BUTYRATE FOR COLON RELEASE

(71) Applicant: BioKier, Inc., Chapel Hill, NC (US)

(72) Inventors: Jerzy Ryszard Szewczyk, Chapel Hill, NC (US); Nicolas D. Kirkland, Norristown, PA (US); Troy A. Bartron, JR., Ardmore, PA (US); Abdul Waseh Basit, Harrow (GB)

(73) Assignee: BioKier, Inc., Chapel Hill, NC (US)

(21) Appl. No.: 16/805,080

(22) Filed: Feb. 28, 2020

(51) Int. Cl. A61K 31/19; A61K 9/50; A61K 45/06

(52) U.S. Cl. CPC A61K 31/19; A61K 45/06; A61K 9/5026; A61K 9/5042

(57) ABSTRACT

The present invention relates to coated butyrate and butyrate plus DPP-IV inhibitor tablets. It has been discovered that an inner butyrate core, a first layer neutral polymer which can dissolve in the colon, followed by an outer coating of a composition, which dissolves only in the colon, prevents the interaction of butyrate with these compositions.

### STATEMENT REGARDING FEDERALLY SPONSORED RESEARCH OR DEVELOPMENT

This invention was partially funded by the National Institutes of Health, government contract number 2R44DK107080-04.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a butyrate tablet and butyrate plus additional active component such as DPP-IV inhibitors, with a coating that insures dissolution in the colon and nowhere else. In particular, it relates to a dual layer coating for butyrate that prevents early dissolution.

### Description of Related Art

The compound butyrate and its salts are well known compositions for treating diabetes, metabolic syndrome, hypertriglyceridemia and obesity, as well as other metabolic disease conditions if it is delivered to the colon and not the upper digestive tract, including the stomach. Its effectiveness can be observed by noting the decrease in glucose present after treatment.

Butyrate, its salts and butyric acid is a naturally occurring fatty acid occurring in the form of esters in animal fats and plant oils. For example, the triglyceride of butyric acid makes up 3% to 4% of butter. It is found in rancid foods, such as butter and cheese, and has a very unpleasant smell and taste. It is an important member of the fatty acid sub-group called the short chain fatty acids.

Obesity is a medical condition that is reaching epidemic proportions among humans in a number of countries throughout the world. It is a condition that is also associated with, or induces other diseases or conditions that disrupt life's activities and lifestyles. Obesity is recognized as a serious risk factor for other diseases and conditions, such as diabetes, hypertension, and arteriosclerosis, and can contribute to elevated levels of cholesterol in the blood. It is also recognized that increased body weight due to obesity can place a burden on joints, such as knee joints, causing arthritis, pain, and stiffness. Obesity can contribute to certain skin conditions, such as atopic dermatitis and bed sores. Because overeating and obesity have become such a problem in the general population, many individuals are now interested in losing weight, reducing weight, and/or maintaining a healthy body weight and lifestyle.

Diabetes mellitus is a worldwide health threat of increasing magnitude, and is considered a major health risk in both developed countries and in developing countries. Type II diabetes accounts for the vast majority of the cases involving diabetes and accounts suggest it is the seventh leading cause of death in the United States. It appears that the major contributing factor to the incidence of Type II diabetes is being overweight. In the United States alone, it is estimated that over 17.6 million individuals suffer from diabetes, and it is estimated that an additional 5.7 million individuals are unaware they have diabetes. In addition, there are about 57 million Americans who are considered pre-diabetic.

Type II diabetes is also known as non-insulin dependent diabetes. It generally manifests itself as an inability to adequately regulate blood-glucose levels. This is as opposed to Type I diabetes which is characterized by defects in pancreatic production of insulin. In other words, it appears that Type II diabetes sufferers suffer from too little insulin or insulin resistance. The factors that have been identified in contributing to these Type II diabetes factors include one or more of obesity, genetic background, age, diet, and blood chemistry. Type II diabetes is frequently called adult-onset, but because diet is a factor, it can arise at virtually any age.

The results of Type II diabetes cause glucose levels to rise in the blood and urine, which in turn can cause hunger, urination, thirst, and metabolism related issues. If the condition is not treated, the most common serious results include heart disease, kidney disease, and blindness. Several treatments are currently being used. Because obesity is frequently a causal agent in diabetes, diet and exercise are usually a front-line defense. Therapeutic agents are also used, usually as a second line of defense, including the use of insulin or pharmaceuticals that reduce glucose levels in blood and urine.

Several drugs are in current use for Type II diabetes, including insulin secretagogues, glucose lowering effectors, GLP-1 analogs, DPP-IV, activators of the peroxisome proliferator activated receptor-gamma, and alpha-glucosidase inhibitors. Because these current treatments have several problems associated with them, there is still a need for alternative therapies to treat Type II diabetes. One new entry in the field is the use of a butyrate to reduce glucose levels and treat metabolic disease when delivered directly to the colon.

However, while there are several compositions known to deliver any drug to the colon, butyrate has its own special problem. It has been discovered that butyric acid interacts with these colon targeted coatings, leaving holes in the coating, and thus causing the tablets to dissolve prematurely. A safer, more reliable colon-targeted delivery of drugs, such as butyrate, is needed.

### BRIEF SUMMARY OF THE INVENTION

The present invention relates to the discovery that putting an intermediate layer of a neutral polymer before coating it with a composition (which only dissolves in the colon) solves the previous problems encountered in coating butyrate for colon delivery.

Accordingly, in one embodiment, there is a tablet of butyrate comprising:
a) a butyrate core;
b) the butyrate core coated with a neutral polymer in a thickness from about 5-100 microns; and
c) the neutral polymer coated with a composition that only dissolves in the colon when the tablet is given orally.

In another embodiment, there is a method of delivering butyrate to the colon of a patient in need thereof and bypassing the upper digestive tract and stomach comprising:
a) formulating a butyrate core;
b) coating the butyrate core with a first layer neutral polymer in a thickness from about 5-100 microns;
c) coating the core and first layer with a composition that only dissolves in the colon to form a coated tablet; and
d) delivering the coated tablet to the patient.

### BRIEF DESCRIPTION OF THE DRAWINGS

FIG. 1 is an electron microscope of a coated butyrate without the intermediate layer of the present invention.

FIG. 2 is a graphic representation of the tablet of the present invention.

FIG. 3 is an electron microscopic image of a coated butyrate with the intermediate layer.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “treating” refers to alleviating the specified condition, eliminating or reducing the symptoms of the condition, slowing or eliminating the progression of the condition and preventing or delaying the initial occurrence of the condition in a subject, or reoccurrence of the condition in a previously afflicted subject.

As used herein, the term “condition or disorder” refers to any disease state, a particular state of a mammal, or the like, to which an increase in the production of a gut hormone from L-cells would affect in a positive or negative way. Included are the disease states noted herein, but in general, this refers to any state so affected by increasing a gut hormone from L-cells in a desired manner. The gut hormone system is known in mammals and as such, the present invention relates to the treatment of a mammal. In one embodiment, the mammal is a human. Conditions for treatment by increasing a gut hormone from L-cells production include, but are not limited to, Type I diabetes, Type II diabetes, obesity, appetite control, metabolic syndrome, and polycystic ovary syndrome.

The gut hormone secretion in the present invention is stimulated in L-cells present in the colon, normally in response to the presence of nutrients in the gut. While such cells are present in other parts of the digestive tract and other parts of the organism, they have the highest concentration in the colon. Stimulation of L-cells in the colon results in the most effective production of gut hormones possible and thus, the most effective treatment. Gut hormones from L-cells of the present invention include, but are not limited to, GLP-1, GP-2, PYY, and oxyntomodulin. Incretins such as GLP-1, in particular, are a gut hormone of interest in one embodiment.

The butyrate compounds of the present invention are for the treatment of diabetes, metabolic syndrome, hypertriglyceridemia, and obesity, as well as other metabolic disease conditions if it is delivered to the colon and not the upper digestive tract, including the stomach. Its effectiveness can be observed by noting the decrease in glucose present after treatment.

The butyrate compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art, such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers, as well as purified enantiomers or enantiomerically/diastereomerically enriched mixtures. Also included within the scope of the present invention are the individual isomers of the compounds, as well as any wholly or partially equilibrated mixtures thereof. The present invention also includes the individual isomers of the compounds represented by the formulas above as mixtures with isomers, thereof, in which one or more chiral centers are inverted.

Typically, but not absolutely, the compounds herein include the salts of the present compositions and include the pharmaceutically acceptable salts. Salts encompassed within the term “pharmaceutically acceptable salts” refer to non-toxic salts of the compounds of this invention. Salts of the compounds of the present invention may include acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, thethiodide, thmethylammonium, and valerate salts. Other salts, which are not pharmaceutically acceptable, may be useful in the preparation of compounds of this invention, and these should be considered to form a further aspect of the invention.

As used herein, the term “administering” of a composition of the present invention refer to oral, in a formulation designed to only deliver the drug to the colon. As described elsewhere herein, the compounds are so formulated to be taken so as to bypass the upper digestive tract and stomach, or rectally to deliver the composition to the colon.

As used herein, the term “effective amount” means that amount of a drug or pharmaceutical agent that will elicit the biological or medical response of a tissue, system, animal, or human that is being sought, for instance, by a researcher or clinician.

As used herein, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes, within its scope, amounts effective to enhance normal physiological function. A therapeutically effective amount will produce a “therapeutic effect”.

For use in therapy, therapeutically effective amounts of a compound of the present invention, as well as salts thereof, are presented as a pharmaceutical composition formulated to release in a colon targeted delivery system, specifically with a neutral first layer and a colon releasing outer layer.

The present invention provides pharmaceutical compositions that include effective amounts of a compound as herein described, or a salt thereof, and one or more pharmaceutically acceptable carriers, diluents, or excipients. The carrier(s), diluent(s), or excipient(s) must be acceptable, in the sense of being compatible with the other ingredients of the formulation and not deleterious to the recipient of the pharmaceutical composition and consistent with the mode of administration, i.e., oral or rectal.

In accordance with another aspect of the present invention, there is also provided a process for the preparation of a pharmaceutical formulation, including admixing a compound of the present invention or salts thereof, with one or more pharmaceutically acceptable carriers, diluents, or excipients.

A therapeutically effective amount of a compound of the present invention will depend upon a number of factors. For example, the species, age, and weight of the recipient, the precise condition requiring treatment and its severity, the nature of the formulation, and the type of colon targeted delivery system, are all factors to be considered. The therapeutically effective amount ultimately should be at the discretion of the attendant, physician, or veterinarian. Regardless, an effective amount of a gut hormone compound of the present invention for the treatment of humans suffering from diabetes or an overweight condition and associated conditions generally should be in the range of 0.01 to 100 mg/kg body weight of recipient (mammal) per day. More often, the effective amount should be in the range of 0.3 to 30 mg/kg body weight per day. Thus, for a 70 kg adult mammal, the actual amount per day would usually be from 21 to 2100 mg. This amount may be given in a single dose per day or in a number (such as two, three, four, five, or more) of sub-doses per day such that the total daily dose is the same. An effective amount of a salt or solvate thereof may be determined as a proportion of the effective amount of the compound of the present invention per se. Similar dosages should be appropriate for treatment of the other conditions referred to herein.

Pharmaceutical formulations of the tablet presented herein are presented in unit dose forms containing a predetermined amount of active ingredient per unit dose. Such a unit may contain as a non-limiting example, 0.5 mg to 1 g of a compound of the present invention depending on the exact condition being treated, and the age, weight, and condition of the patient. Preferred unit dosage formulations are those containing a daily dose or sub-dose, as herein above recited or an appropriate fraction thereof, of an active ingredient. Such pharmaceutical formulations may be prepared by any of the methods well known in the pharmacy art.

The compounds of the present invention, or a salt thereof, are administered by a colon targeted coating drug delivery system. In one embodiment, the delivery systems may be employed for targeting drug delivery to the colon and bypassing the upper digestive system and stomach. Such drug delivery systems include coatings of the outer layer with one or more covalent linkage compositions, polymer coated compositions, compositions embedded in matrices, time released compositions, redox-sensitive polymer compositions, bioadhesive compositions, micropartical coating compositions, and osmotic delivery compositions. Suitable compositions include those containing polysaccharides, such as chitosan, pectin, chondroitin sulphate, cyclodexthn, dextrans, guar gum, inulin, amylose, and locust bean gum. The compounds may also be coupled with soluble polymers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. Those of particular effectiveness in the present invention include embodiments of multi-matrix targeted systems. Of particular effectiveness in the present invention are the targeted matrix in matrix systems comprising a formulation of a hydrophilic first matrix, comprising a lipophilic phase and an amphiphilic phase, wherein the lipophilic phase and the amphiphilic phase are in a second matrix together, and the second matrix is dispersed throughout the hydrophilic first matrix, and wherein the pharmaceutical composition containing the compound is at least partially incorporated into the amphiphilic phase. Examples of some of the matrix in matrix formulations are disclosed in U.S. Pat. No. 7,431,943, issued on Oct. 7, 2008, in the name of Villa et al. noted above. Those skilled in the art will appreciate the use of such compositions for the purposes of targeting delivery of the compounds of the present invention, or a salt thereof, to the colon of the subject being treated.

The compounds of the present invention or a salt thereof may be employed alone or in combination with other therapeutic agents. The compound(s) of the present invention and the other pharmaceutically active agent(s) may be administered together in the same tablet or administered in separate tablets or when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention or a salt or solvate thereof with other treatment agents may be in combination by administration concomitantly in: (1) a unitary pharmaceutical composition, including both compounds; or (2) separate pharmaceutical compositions, each including one of the compounds. Alternatively, the combination may be administered separately in a sequential manner, wherein one treatment agent is administered first and the other second, or vice versa. Such sequential administration may be close in time or remote in time.

The compounds are formulated in compositions comprising a butyrate core, a neutral polymer first layer and a colon releasing outer layer. The compositions so formulated will be designed to give an effective dosage to the colon. The first layer is applied at a thickness of about 5-100 microns. In one embodiment, the thickness of the first layer is 10-50 microns.

The compounds of the present invention may be used in the treatment of a variety of disorders and conditions. As such, the compounds of the present invention may be used in combination with a variety of other therapeutic agents useful in the treatment of those disorders or conditions. As discussed briefly above, current diabetes therapies include diet, exercise, insulin, insulin secretagogues, glucose-lowering effectors, PPAR-γ agonists, and α-glucosidase inhibitors. The compounds of the present invention may be combined with these or other medical therapies to treat and/or prevent diabetes and associated disorders and conditions, including but not limited to, diabetes Types I and II, obesity, glucose intolerance, insulin resistance, metabolic syndrome, hyperlipidemia, hypercholesterolemia, atherosclerosis, neurodegenerative diseases, and other indications such as inflammation and stroke. For example, in the treatment of Type II diabetes, a compound of the present invention may be combined with one or more pharmaceutically active agents, including metformin, sulfonylureas such as glyburide and glipizide, repaglinide, nateglinide, thiazolidinediones such as rosiglitazone and pioglitazone, acarbose, miglitol, exanatide, pramlintide, and insulin.

The butyrate tablets of the present invention are formulated by first creating a core of butyrate or butyrate plus additional active compounds (e.g., a DPP-IV Inhibitor). From there, the core is coated with a neutral polymer before the addition of an outer coat of a composition, which only dissolves in the colon as described above. Coating is by those means known in the pharmaceutical art. One example is dual-trigger enteric coating. The neutral polymer is 5-100 microns thick and the outer coat at 50-250 microns thick.

As used herein, the term “neutral polymer” refers to hydroxypropyl methylcellulose (HPMC) type polymers, gelatin, etc. The neutral polymer should have a thickness of 5 to 100 microns such that it dissolves in the colon after the outer layer is removed. In one embodiment, the polymer coating is from 0.5% to about 10% the weight of the butyrate core. In one embodiment, it is Opadry® Clear (PVA-based, polyvinyl alcohol).

Example 1 is the manufacture of the butyrate tablet of the present invention.

### Tablet Manufacture Examples

### Sodium Butyrate Tablets, Formulation, and Process

### Example 1

### Granulation Procedure

1. Hypromellose (Pharmacoat 603) is mixed with water to produce a binder solution.
2. The remaining dry components are added to a top-drive high shear granulator and pre-mixed.
3. Binder solution is added to the granulator bowl while mixing continues.
4. Water is added to the granulation until the target endpoint is reached.
5. The granulation is passed through a conical mill to remove large lumps.
6. The de-lumped granulation is added to a fluid bed and dried to below 2% moisture content.
7. The dried granulation is sized using a conical mill.

### Example 2

### Granulation Formulation

Binder Solution Formulation

| Component | % w/w | mg/dose |
|---|---|---|
| Pharmacoat 603 | 12.0% | 30.0 |
| Water | 88.0% | 0.0 |
| Total | 100.0% | 30.0 |

Granulation Formulation

| Component | % w/w | mg/dose |
|---|---|---|
| Sodium Butyrate | 58.8% | 500.0 |
| Pharmacoat 603 | 3.5% | 30.0 |
| Methocel K100 Premium CR | 16.5% | 140.0 |
| Prosolv SMCC 50 | 21.2% | 180.0 |
| Total | 100.0% | 850.0 |

### Example 3

### Blending and Compression Procedure

1. The milled granulation is combined with additional excipients using a v-blender.
2. The blend is compressed into tablets on a rotary tablet press using D-type tooling using the following targets:

B:

| Parameters | Specification |
|---|---|
| Tablet Weight | 1350 ± 5.0% mg |
| Tablet Hardness | 14-26 kp |
| Friability | NMT 1.0% |

Blend Formulation

Tablet Blend Formulation

| Component | % w/w | mg/dose |
|---|---|---|
| Dried Granulation | 63.0% | 850.0 |
| Prosolv SMCC 90 | 36.1% | 488.0 |
| Magnesium Stearate | 0.9% | 12.0 |
| Total | 100.0% | 1350.0 |

### Example 4

### Coating

1. Tablet cores are sub-coated in a perforated coating pan with clear polymer coat to 1.5% of target weight gain.
2. Sub-coated cores are coated with dual-trigger enteric coating to 5% of target weight gain.

C.

Sub-Coating Solution Formulation

| Material | % w/w |
|---|---|
| Opadry Clear | 10% |
| Sterile Water | 90% |
| Total | 100% |

Phlora Coating Complete Solution Formulation

| | Solids | |
|---|---|---|
| Material | Content | % w/w |
| Maize Starch Amylo N-460 | 100% | 1.48% |
| Eudragit S 100 | 100% | 3.45% |
| Plasacryl T20 | 20% | 4.92% |
| TEC | 100% | 0.74% |
| Butanol | 0% | 2.95% |
| Ethanol200 Proof | 0% | 51.28% |
| Water | 0% | 35.18% |
| Total | 6.6% | 100.00% |

| Component | % w/w | mg/dose |
|---|---|---|
| Sodium Butyrate | 34.8% | 500.0 |
| Pharmacoat 603 | 2.1% | 30.0 |
| Methocel K100 Premium CR | 9.7% | 140.0 |
| Prosolv SMCC 50 | 12.5% | 180.0 |
| Prosolv SMCC 90 | 33.9% | 488.0 |
| Magnesium Stearate | 0.8% | 12.0 |
| Opadry Clear | 1.4% | 20.3 |
| Maize Starch Amylo N-460 | 1.1% | 15.2 |
| Eudragit S 100 | 2.5% | 35.5 |
| Plasacryl T20 | 0.7% | 10.2 |
| TEC | 0.5% | 7.6 |
| Total | 100.0% | 1438.8 |

### DRAWINGS

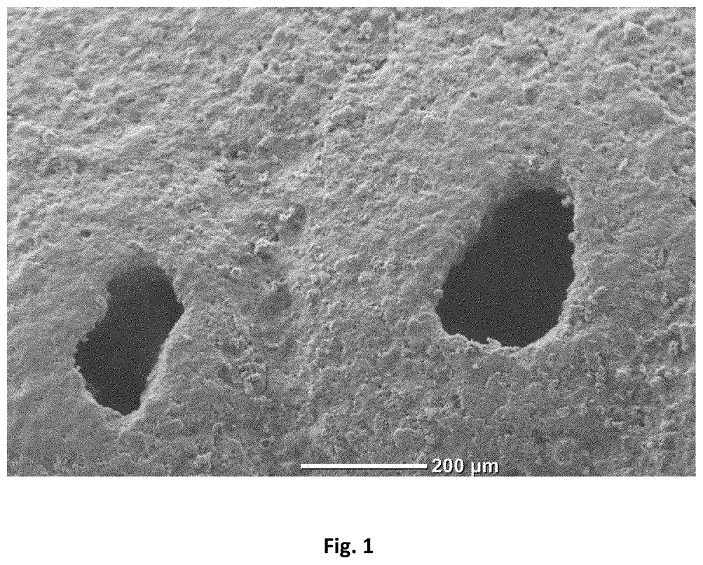

Now referring to the drawings, FIG. 1 is an electron microscopic picture of a butyrate core only covered with the outer layer of the present invention, in this case a dual-trigger enteric coating. It is noted that there are multiple holes in the coating which cause the tablet to at least partially dissolve before reaching the colon.

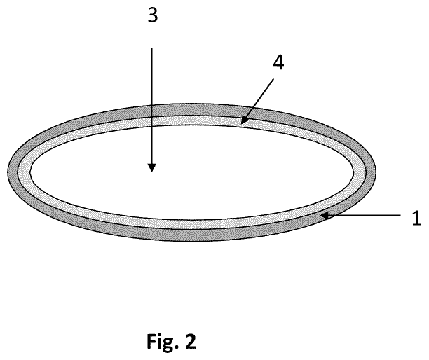

FIG. 2 is a cross section of an embodiment of the tablet of the present invention. It shows the butyrate core 3, a first coating of a neutral polymer 4, and an outer layer of a composition that only dissolves in the colon 1.

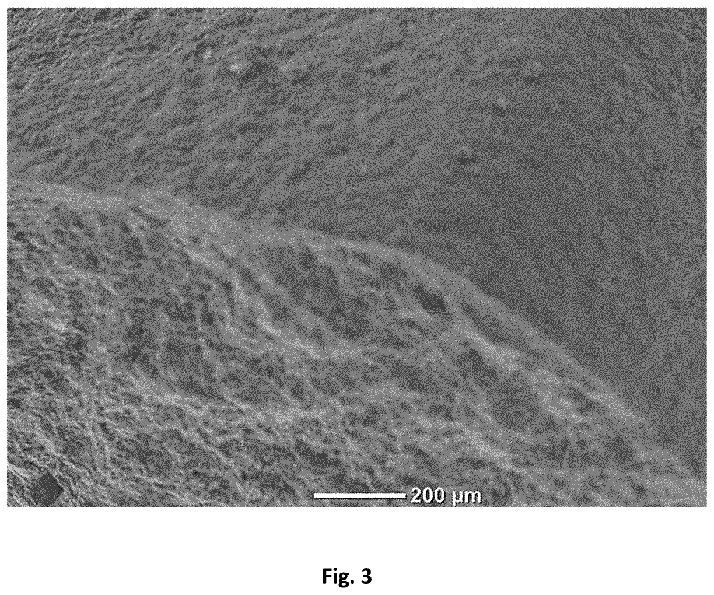

FIG. 3 is an electron microscopic picture of the outer coating of a tablet having the construction disclosed herein. It can easily be seen that it is a smooth surface without the holes evident in FIG. 1.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A tablet of butyrate comprising:
a) a butyrate core;
b) the butyrate core coated with a neutral polymer in a thickness from about 5-100 microns; and
c) the neutral polymer coated with a composition that only dissolves in the colon when the tablet is given orally.

2. The tablet of butyrate according to claim 1 wherein the neutral polymer is selected from the group consisting of: a polyvinyl alcohol and a methylcellulose polymer.

3. The tablet of butyrate according to claim 1 wherein the composition that only dissolves in the colon is a dual-trigger enteric coating.

4. The tablet of butyrate according to claim 1 wherein the neutral polymer layer has a thickness of from about 10-50 microns.

5. The tablet of butyrate according to claim 1 wherein the butyrate core further comprising one or more additional medically active components.

6. The tablet of butyrate according to claim 1 wherein the additional medically active component is a DPP-IV inhibitor.

7. A method of delivering butyrate to the colon of a patient in need thereof and bypassing the upper digestive tract and stomach comprising:
a) formulating a butyrate core;
b) coating the butyrate core with a first layer neutral polymer in a thickness from about 5-100 microns;
c) coating the core and first layer with a composition that only dissolves in the colon to form a coated tablet; and
d) delivering the coated tablet to the patient.

8. The method of delivering butyrate to the colon of a patient according to claim 7 wherein the patient is suffering from at least one of diabetes, metabolic syndrome, hypertriglyceridemia obesity, and any other metabolic disease.

9. The method of delivering butyrate to the colon of a patient according to claim 7 wherein the neutral polymer layer has a thickness of from about 10-50 microns.

10. The method of delivering butyrate to the colon of a patient according to claim 7 wherein the butyrate core further comprising one or more additional medically active components.

11. The method of delivering butyrate to the colon of a patient according to claim 7 wherein the additional medically active component is a DPP-IV inhibitor.
